(19) United States
(12) Patent Application Publication
(10) Pub. No.: US 2026/0184680 A1
(43) Pub. Date: Jul. 2, 2026

(54) METHOD FOR SIMULTANEOUSLY PREPARING CHLOROPHYLL PASTE AND 1-DEOXYNOJIRIMYCIN

(71) Applicant: HENAN ZHONGDA HENGYUAN BIOTECHNOLOGY STOCK CO., LTD., Luohe City (CN)

(72) Inventors: Yihong LIU, Luohe City (CN); Ziheng JIN, Luohe City (CN); Yanjun WEN, Luohe City (CN); Tianyi PAN, Luohe City (CN); Huipeng PEI, Luohe City (CN); Sujing GU, Luohe City (CN); Yang LIU, Luohe City (CN); Xuejiao CHEN, Luohe City (CN)

(21) Appl. No.: 19/094,159

(22) Filed: Mar. 28, 2025

(30) Foreign Application Priority Data
Dec. 27, 2024 (CN) 202411945219.9

(51) Int. Cl. C07D 211/46; B01D 11/02; C07F 3/02

(52) U.S. Cl. CPC C07D 211/46; B01D 11/0288; C07F 3/02

(57) ABSTRACT

A method for simultaneously preparing a chlorophyll paste and 1-deoxynojirimycin includes the following steps: (1) drying a silkworm excrement in a shaded area until the silkworm excrement reaches a specific moisture content; (2) placing the silkworm excrement into an extraction tank, adding an extraction solvent to obtain a mixture, and stirring the mixture with a water bath, filtering the mixture, the extraction solvent comprising both a non-polar solvent and a polar solvent; (3) subjecting the mixture to a coarse filtration to obtain a crude filtrate; (4) subjecting the crude filtrate to centrifugation, obtaining a chlorophyll-rich light phase and a 1-deoxynojirimycin-rich heavy phase; and (5) undergoing fine filtration and concentration with the chlorophyll-rich light phase to obtain a chlorophyll paste, and subjecting the 1-deoxynojirimycin-rich heavy phase with fine filtration, concentration, formulation, and spray drying to prepare 1-deoxynojirimycin.

This application claims priority to Chinese Patent Application No. 202411945219.9, filed on Dec. 27, 2024, which is incorporated by reference for all purposes as if fully set forth herein.

### TECHNICAL FIELD

The present invention belongs to the field of food additive refinement technology, and specifically relates to a method for simultaneously preparing a chlorophyll paste and 1-deoxynojirimycin.

### BACKGROUND

Silkworm excrement is the excrement of silkworms after they feed on mulberry leaves. It is not only rich in crude protein and carbohydrates but also can be used to extract chlorophyll, lutein, carotenoids, plant alcohols, leaf proteins, pectin, 1-deoxynojirimycin, and other compounds. It has broad applications in the fields of food, pharmaceuticals, feed, and daily chemicals. Literature shows that the chlorophyll content in silkworm excrement can reach as high as 0.8-1.2%.

Silkworm excrement is the excrement of silkworms after they feed on mulberry leaves. It is not only rich in crude protein and carbohydrates but also can be used to extract chlorophyll, lutein, carotenoids, plant alcohols, leaf proteins, pectin, 1-deoxynojirimycin, and other compounds. It has broad applications in the fields of food, pharmaceuticals, feed, and daily chemicals. Literature shows that the chlorophyll content in silkworm excrement can reach as high as 0.8-1.2%.

Existing literature, such as “The Effect of Different Solvents on the Extraction of Chlorophyll from Silkworm Excrement,” “Study on the Extraction Process of Chlorophyll from Silkworm Excrement,” “Research on the Extraction and Properties of Chlorophyll from Silkworm Excrement,” “Study on the Process Parameters of Ultrasound-Assisted Extraction of Chlorophyll from Silkworm Excrement,” and “Study on the Extraction of Chlorophyll from Silkworm Excrement Using Mixed Solvents and the Utilization of Residual Materials” (Li Jing), provides detailed reports on the extraction solvents and methods for chlorophyll from silkworm excrement. Additionally, technological achievements such as “Application of Countercurrent Technology in the Extraction of Chlorophyll from Silkworm Excrement” and “A Continuous Method for the Extraction of Chlorophyll from Silkworm Excrement” describe methods using continuous countercurrent extraction for chlorophyll extraction from silkworm excrement. The methods and solvents mentioned above have all achieved certain success in extracting chlorophyll from silkworm excrement.

It is well known that silkworm excrement contains a variety of active compounds, all of which have a wide range of applications in the market. However, existing technologies focus solely on the extraction of single components from silkworm excrement. In practical production, the emphasis is often placed on the extraction efficiency and quality of individual components, while the comprehensive development of multiple substances is neglected. Furthermore, the prior art does not disclose a method for the high-efficiency, high-quality simultaneous extraction of two or more components from silkworm excrement.

In summary, providing a method for the comprehensive development and utilization of silkworm excrement is a problem that urgently needs to be addressed by those skilled in the art.

### SUMMARY OF THE INVENTION

The objective of the present invention is to provide a method for the simultaneous preparation of high-purity chlorophyll paste and 1-deoxynojirimycin. The technical solution provided by the present invention uses fresh silkworm excrement with a specific moisture content as the raw material and employs a mixed solvent extraction method combining polar and non-polar solvents. This method allows for the simultaneous extraction of chlorophyll and 1-deoxynojirimycin into the same extraction solution. The extraction solution is then subjected to centrifugation, yielding a light phase containing chlorophyll and a heavy phase containing 1-deoxynojirimycin. The light phase is refined through filtration and reduced-pressure concentration to obtain a chlorophyll paste product with a content greater than 27%, an extraction yield of over 95%, and an average yield of 0.59%; the 1-deoxynojirimycin in the heavy phase also undergoes the removal of lipid-soluble impurities during the phase separation process. After separation, concentration, and drying, a water-soluble 1-deoxynojirimycin powder product with a content of more than 3% is obtained, with an average yield of 2.88%

The technical solution provided by the present invention, through the design of solvents and extraction processes, uses fresh silkworm excrement as the raw material and a single mixed extractant to efficiently and simultaneously extract both chlorophyll and 1-deoxynojirimycin. Compared to the prior art, this method not only increases the chlorophyll paste content, providing high-quality raw materials for the preparation of microencapsulated chlorophyll, oil-soluble chlorophyll copper, and chlorophyll copper sodium salts, but also yields high-quality water-soluble 1-deoxynojirimycin, offering a new approach and effective method for the comprehensive development and utilization of silkworm excrement.

To achieve the above objectives, the present invention provides a method for the simultaneous preparation of high-purity chlorophyll paste and 1-deoxynojirimycin, comprising the following steps:
(1) dry the fresh silkworm excrement in a shaded area until it reaches a specific moisture content;
(2) place the dried silkworm excrement into the extraction vessel, add the extraction solvent, and stir under water bath conditions to extract. Filter the mixture to obtain a mixed solution; wherein, the extraction solvent is composed of a non-polar solvent and a polar solvent;
(3) the obtained mixture is then subjected to coarse filtration to yield a crude filtrate;
(4) the obtained crude filtrate is then subjected to centrifugation to separate into a light phase containing chlorophyll and a heavy phase containing 1-deoxynojirimycin;
(5) the light phase containing chlorophyll is then subjected to fine filtration and concentration to obtain high-purity chlorophyll paste. The heavy phase containing 1-deoxynojirimycin is subjected to fine filtration, concentration, formulation, and spray drying to obtain 1-deoxynojirimycin.

In a preferred embodiment, in step (1), the moisture content of the dried silkworm excrement is 40-60%. Currently, the traditional method for processing silkworm excrement involves drying or sun-drying to reduce the moisture content to below 20% for long-term storage. Prior to extraction, water is added to soften the silkworm excrement to a moisture content of 40-60%. However, both the drying and the pre-extraction softening treatment of silkworm excrement take a long time, and the process generates heat, which leads to the loss of chlorophyll. Furthermore, the process of first reducing the moisture and then rehydrating it for softening is repetitive, which increases the cost. In the present invention, fresh silkworm excrement is dried to a moisture content of 40-60%, and extraction is directly performed, which not only simplifies the process but also reduces the loss of active components.

In a preferred embodiment, in step (2), the weight ratio of the non-polar solvent to the polar solvent is (1˜10):1, and preferably, the weight ratio of the non-polar solvent to the polar solvent is (2˜6):1.

In a preferred embodiment, in step (2), the non-polar solvent comprises ether solvents and/or alkane solvents; the polar solvent is a small molecule alcohol.

In a preferred embodiment, in step (2), the non-polar solvent comprises one or more of petroleum ether, ether, methyl tert-butyl ether, and alkanes with 4 to 7 carbon atoms; the polar solvent comprises one or more of methanol, ethanol, and isopropanol.

In a preferred embodiment, in step (2), the solid-to-liquid ratio of silkworm excrement to the extraction solvent is 1:(2˜20); preferably, the solid-to-liquid ratio of silkworm excrement to the extraction solvent is 1:(3˜10).

In a preferred embodiment, in step (2), the water bath temperature is 30˜60° C.; the extraction time is 0.5˜5 hours; preferably, the water bath temperature is 40˜60° C.; the extraction time is 2˜4 hours.

In a preferred embodiment, in step (2), the number of extraction cycles is 1-8; preferably, only the mixed solution from the first extraction is used for coarse filtration in step (3), the remaining mixed solutions from subsequent extractions can be reused as extraction solvent, after replenishing with the appropriate ratio of non-polar and polar solvents, these solutions can be used for the extraction of additional batches of fresh silkworm excrement, thereby preventing waste liquid generation.

In a preferred embodiment, in step (3), the mesh size of the coarse filtration device is 100˜500 mesh. The specific device may be any conventional equipment known to those skilled in the art, such as pipeline filters, leaf filters, or bag filters. Preferably, the mesh size of the coarse filtration device is 300˜500 mesh.

In a preferred embodiment, in step (4), the centrifugation device includes explosion-proof centrifuges suitable for liquid-liquid phase separation containing organic solvents, such as disc centrifuges, tubular centrifuges, and scroll centrifuges.

In a preferred embodiment, in step (5), the mesh size of the fine filtration device for the chlorophyll-containing light phase is 500-1500 mesh. The specific device may be any conventional equipment known to those skilled in the art, such as bag filters, cardboard filters, or plate-frame filters. Preferably, the mesh size of the fine filtration device for the chlorophyll-containing light phase is 800˜1200 mesh.

In a preferred embodiment, in step (5), the concentration conditions for the fine filtrate containing chlorophyll include: concentration temperature of 40˜70° C., concentration pressure of −0.02 to −0.09 MPa, and concentration until the residual solvent volume in the chlorophyll-containing light phase is less than 1%. Preferably, the concentration conditions for the fine filtrate containing chlorophyll include: concentration temperature of 60˜70° C., concentration pressure of −0.04 to −0.09 MPa.

In a preferred embodiment, in step (5), the high-purity chlorophyll paste obtained has a content of more than 27%, an extraction rate of more than 95%, and an average yield of 0.59%.

In a preferred embodiment, in step (5), the mesh size of the fine filtration device for the heavy phase containing 1-deoxynojirimycin is 500˜1500 mesh. The specific device may be any conventional equipment known to those skilled in the art, such as bag filters, cardboard filters, or plate-frame filters. Preferably, the mesh size of the fine filtration device for the heavy phase containing 1-deoxynojirimycin is 800˜1200 mesh.

In a preferred embodiment, in step (5), the concentration conditions for the fine filtrate containing 1-deoxynojirimycin include: concentration temperature of 40-100° C., concentration pressure of −0.02 to −0.09 MPa, and concentration to a Brix value of 10-40%. Preferably, the concentration conditions for the fine filtrate containing 1-deoxynojirimycin include: concentration temperature of 60-90° C., concentration pressure of −0.06 to −0.09 MPa, and concentration to a Brix value of 20-30%.

In a preferred embodiment, in step (5), the auxiliary materials for formulation include one or more of maltodextrin, arabic gum, and modified starch, and the amount of the auxiliary materials is 5-20% by weight of the concentrated solution containing 1-deoxynojirimycin.

In a preferred embodiment, in step (5), the spray drying conditions for the formulation liquid containing 1-deoxynojirimycin include: an inlet temperature of 160˜220° C. and an outlet temperature of 80˜120° C.; more preferably, the spray drying conditions for the formulation liquid containing 1-deoxynojirimycin include: an inlet temperature of 180˜210° C. and an outlet temperature of 90˜120° C.

In a preferred embodiment, in step (5), the 1-deoxynojirimycin obtained has a content of more than 3%, with an average yield of 2.88%.

In a preferred embodiment, in step (5), there is no particular order for the preparation of high-purity paste chlorophyll and 1-deoxynojirimycin; both preparing high-purity paste chlorophyll first or preparing 1-deoxynojirimycin first are within the scope of the present invention.

Another objective of the present invention is to provide high-purity paste chlorophyll and/or 1-deoxynojirimycin prepared by any of the above methods.

Compared with the prior art, the technical solution of the present invention offers the following advantages:

In the present invention, a method of mixed extraction using both polar and non-polar solvents is employed. This approach not only leverages the penetration ability of the polar solvent and the solubilizing capacity of the non-polar solvent to enhance the extraction yield of chlorophyll, but also enables the extraction of another polar compound, 1-deoxynojirimycin, thereby achieving a comprehensive utilization of the materials. Additionally, during the centrifugation process of the extraction solution, not only are the two products effectively separated, but the dewatering process of the paste-like chlorophyll is also completed. Following phase separation, the chlorophyll is present in the non-polar solvent phase, and high-purity paste-like chlorophyll can be obtained after solvent recovery, eliminating the need for further dewatering procedures.

In this invention, fresh silkworm excrement is used as raw material for the first time. After drying to a specific moisture content, they undergo a combined extraction process using polar and non-polar solvents. The obtained extraction solution is coarsely filtered and then subjected to centrifugation to simultaneously yield a chlorophyll extract solution and a 1-deoxynojirimycin extract solution. The chlorophyll extraction solution, after fine filtration and concentration, results in a paste-like chlorophyll product with a content exceeding 27%, an extraction rate above 95%, and an average yield of 0.59%. Meanwhile, the 1-deoxynojirimycin extraction solution, after fine filtration, concentration, blending, and drying, produces a 1-deoxynojirimycin powder product with a content exceeding 3% and an average yield of 2.88%.

The overall process of this invention is straightforward and highly efficient, eliminating the need for the softening step of silkworm excrement and resolving the issue in traditional methods where paste-like chlorophyll requires additional dewatering. This not only shortens the operation time but also significantly improves the yield of paste-like chlorophyll. By utilizing a single-step extraction method with the same solvent, two products are obtained simultaneously, thereby enhancing the value of silkworm excrement and providing an effective solution for its comprehensive development and utilization.

### DETAILED DESCRIPTION OF EMBODIMENTS

To facilitate a deeper understanding of the present invention by those skilled in the art, the following provides a more detailed explanation in conjunction with specific embodiments. However, it should be understood that the scope of the invention is not limited to these particular embodiments.

The embodiments of the present invention provide a method and products for simultaneously producing high-purity paste-like chlorophyll and 1-deoxynojirimycin, addressing the issues in existing technologies, including complex processes, low product purity, low raw material utilization, and the inability to obtain two high-quality products within a single process.

The technical solutions of the present application are described in detail below through specific embodiments:

Unless otherwise specified, the technical methods employed in the present invention are conventional methods well known to those skilled in the art. The various raw materials, reagents, instruments, and equipment used in the present invention can be commercially obtained or prepared using established methods. Unless specifically indicated otherwise, all reagents used in the present invention are of analytical grade.

### Example 1

(1) The fresh silkworm excrement, after drying, had a moisture content of 55.7%. Testing revealed that the chlorophyll content was 0.570%, and the 1-deoxynojirimycin content was 1.084% 0;
(2) Take 500 kg of fresh silkworm excrement treated according to step (1) and placed them into an extraction tank. Add 500 kg of 95% ethanol and 2000 kg of plant oil extraction solvent. The mixture was stirred and extracted four times at a temperature of 58° C., each lasting 2 hours. The extract from the first cycle was filtered through a 500-mesh inline filter to obtain a crude filtrate. After extraction, the silkworm excrement residue contained 0.04% chlorophyll, had a moisture content of 26.64%, and achieved an extraction yield of 95.93%. The extraction liquids from the second to fourth extractions were used for extracting other batches of fresh silkworm excrement;
(3) The crude extraction filtrate was separated using a two-phase liquid-liquid disk separator, yielding a plant oil extraction solvent phase containing chlorophyll and an ethanol phase containing 1-deoxynojirimycin;
(4) The chlorophyll-containing plant oil extraction solvent phase was filtered using an 800-mesh bag filter to obtain a chlorophyll-enriched refined filtrate. The refined filtrate was concentrated under conditions of 58˜65° C. and −0.06 to −0.07 MPa until the solvent residue was less than 1%, yielding 9.54 kg of paste-like chlorophyll with a chlorophyll content of 28.63% and a yield of 0.546%;
(5) The ethanol phase containing 1-deoxynojirimycin was filtered using a 1000-mesh plate and frame filter to obtain a refined filtrate containing 1-deoxynojirimycin. The refined filtrate was then concentrated at 75˜80° C. and −0.07 to −0.08 MPa until reaching a Brix value of 22.40%, yielding 54.79 kg of concentrate with a 1-deoxynojirimycin content of 0.92%;
(6) Maltodextrin was added to the concentrate in an amount equal to 6.57% of the concentrate's weight. The mixture was stirred at 80° C. until completely dissolved and evenly blended, resulting in a 1-deoxynojirimycin mixed solution; the mixed solution was spray-dried at an inlet temperature of 180˜190° C. and an outlet temperature of 80˜90° C., yielding 15.96 kg of 1-deoxynojirimycin powder with a content of 3.09% and a yield of 3.19%.

### Example 2

(1) The fresh silkworm excrement, after drying, had a moisture content of 45.3%. Testing revealed that the chlorophyll content was 0.645%, and the 1-deoxynojirimycin content was 0.936%;
(2) Take 500 kg of fresh silkworm excrement treated according to step (1) and placed them into an extraction tank. Add 800 kg of anhydrous methanol and 2400 kg of petroleum ether. The mixture was stirred and extracted at 54° C. for five times, each lasting 2 hours. The extract from the first extraction was filtered through a 500-mesh inline filter to obtain the crude filtrate. After extraction, the residual silkworm excrement contained 0.03% chlorophyll, had a moisture content of 21.3%, and achieved an extraction yield of 96.77%. The extracts from the second to fifth extractions were used for extracting other batches of fresh silkworm excrement;
(3) The crude extraction filtrate was separated using a liquid-liquid two-phase disk separator, yielding a petroleum ether phase containing chlorophyll and a methanol phase containing 1-deoxynojirimycin;
(4) The chlorophyll-containing petroleum ether phase was filtered using a 1000-mesh plate and frame filter to obtain a refined chlorophyll filtrate. This refined filtrate was then concentrated under conditions of 55˜60° C. and −0.07 to −0.08 MPa until the solvent residue was less than 1%, yielding 9.84 kg of chlorophyll paste with a chlorophyll content of 31.45% and a yield of 0.619%;
(5) The methanol phase containing 1-deoxynojirimycin was filtered using an 800-mesh cardboard filter to obtain a refined filtrate. The refined filtrate was then concentrated under conditions of 73˜78° C. and −0.07 to −0.08 MPa until it reached a Brix value of 25.76%, yielding 35.1 kg of concentrate with a 1-deoxynojirimycin content of 1.28%;
(6) A mixture of gum arabic and maltodextrin (in a 1:3 weight ratio) was added to the concentrate in an amount equal to 15.67% of the concentrate's weight, the mixture was stirred at 80° C. until fully dissolved and evenly blended, resulting in a 1-deoxynojirimycin solution. This solution was spray-dried at an inlet temperature of 185˜195° C. and an outlet temperature of 85˜95° C., yielding 14.1 kg of 1-deoxynojirimycin powder with a content of 3.14% and a yield of 2.82%;

### Example 3

(1) The fresh silkworm excrement, after drying, had a moisture content of 53.2%. The chlorophyll content was determined to be 0.628%, while the 1-deoxynojirimycin content was measured at 0.899%;
(2) Take 500 kg of fresh silkworm excrement treated according to step (1) and placed them into an extraction tank. Add 600 kg of 90% methanol and 1800 kg of plant oil extraction solvent. The mixture was stirred and extracted at 60° C. for five times, each lasting 3 hours. The extract from the first cycle was filtered through a 500-mesh inline filter to obtain a crude filtrate. After extraction, the silkworm excrement residue contained 0.05% solids, had a moisture content of 24.1%, and achieved an extraction rate of 95.09%. The extracts from the second to fourth cycles were used for extracting other batches of fresh silkworm excrement;
(3) The crude extraction filtrate was separated using a liquid-liquid two-phase disk separator, yielding a plant oil extraction solvent phase containing chlorophyll and a methanol phase containing 1-deoxynojirimycin;
(4) The chlorophyll-containing plant oil extraction solvent phase was filtered using a 1000-mesh plate and frame filter to obtain a refined chlorophyll filtrate. The refined filtrate was then concentrated under conditions of 55˜60° C. and −0.07 to −0.08 MPa until the solvent residue was less than 1%, yielding 10.90 kg of chlorophyll paste with a chlorophyll content of 27.84% and a yield of 0.607%;
(5) The methanol phase containing 1-deoxynojirimycin was filtered using a 1200-mesh bag filter to obtain a refined filtrate. The refined filtrate was then concentrated under conditions of 75-80° C. and −0.07 to −0.08 MPa until it reached a Brix value of 18.27%, yielding 54.1 kg of concentrate with a 1-deoxynojirimycin content of 0.79%;
(6) A modified starch was added to the concentrate in an amount equal to 6.47% of the concentrate's weight. The mixture was stirred at 80° C. until fully dissolved and evenly blended, resulting in a 1-deoxynojirimycin solution. This solution was spray-dried at an inlet temperature of 190-195° C. and an outlet temperature of 90-100° C., yielding 13.1 kg of 1-deoxynojirimycin powder with a content of 3.20% and a yield of 2.63%.

### Comparative Example 1

(1) The fresh silkworm excrement, after drying, had a moisture content of 53.2% and a chlorophyll content of 0.628%;
(2) Take 500 kg of fresh silkworm excrement treated according to step (1) and placed them into an extraction tank. Add 2500 kg of 97% acetone solvent. The mixture was stirred at 45° C. and extracted four times, each lasting 2 hours. The extract from the first cycle was filtered through a 500-mesh inline filter to obtain the crude filtrate. After extraction, the silkworm excrement residue contained 0.12% solids, had a moisture content of 24.1%, and achieved an extraction rate of 88.65%;
(3) The chlorophyll-containing crude filtrate was filtered using a 1000-mesh bag filter to obtain a refined chlorophyll filtrate. The refined filtrate was then concentrated at 55˜60° C. and −0.07 to −0.08 MPa until the acetone concentration was less than 10%, resulting in a concentrated solution;
(4) The concentrated solution was allowed to stand at room temperature for phase separation. The lower aqueous phase was slowly drained off, and the upper oily phase was transferred to a dehydration tank. Under conditions of 60˜65° C. and −0.08 to −0.09 MPa, vacuum dehydration was performed for 3.5 hours, yielding 21.08 kg of paste-like chlorophyll with a chlorophyll content of 12.38% and a yield of 0.522%;

### Comparative Example 2

(1) The fresh silkworm excrement, after drying, had a moisture content of 53.2% and a chlorophyll content of 0.628%;
(2) After sun-drying to a moisture content of 14.36%, 320 kg of dried silkworm excrement were obtained, with a chlorophyll content of 0.891%;
(3) 200 kg of water was added to the dried silkworm excrement, stirred and mixed, and then left to soften at room temperature for 4 hours. The mixture was placed to an extraction tank, and 3000 kg of 97% acetone solvent was added. The material was stirred and extracted at 42° C. for four cycles, each lasting 2 hours. The extract from the first cycle was filtered through a 500-mesh inline filter to obtain the crude filtrate. After extraction, the silkworm excrement residue contained 0.09% solids, had a moisture content of 19.7%, and achieved an extraction rate of 89.23%.
(4) The chlorophyll-containing crude filtrate was filtered using an 800-mesh bag filter to obtain a refined chlorophyll filtrate. The refined filtrate was then concentrated at 55˜60° C. and −0.07 to −0.08 MPa until the acetone concentration was below 10%, resulting in a concentrated solution;
(5) The concentrated solution was allowed to stand at room temperature to separate into layers. The lower aqueous phase was slowly drained off, and the upper oily phase was transferred to a dehydration tank. Under conditions of 60˜65° C. and −0.08 to −0.09 MPa, vacuum dehydration was performed for 4 hours, yielding 23.13 kg of paste-like chlorophyll with a chlorophyll content of 10.96% and a yield of 0.507%.

TABLE 1
Comparative analysis of the analytical results for Examples 1-3 and Comparative Examples 1-2.

| | | Experiment number | | | | |
|---|---|---|---|---|---|---|
| | | Example | Example | Example | Comparative | Comparative |
| Test items | | 1 | 2 | 3 | Example 1 | Example 1 |
| Raw material | chlorophyll | 0.570 | 0.645 | 0.628 | 0.628 | 0.628 |
| | content/% | | | | | |
| | moisture/% | 55.7 | 45.3 | 53.2 | 53.2 | 53.2 |
| | 1- | 1.084 | 0.936 | 0.899 | 0.899 | 0.899 |
| | deoxynojirimycin/‰ | | | | | |
| Chlorophyll | chlorophyll | 28.63 | 31.45 | 27.84 | 12.38 | 10.96 |
| paste | content/% | | | | | |
| | moisture/% | 3.87 | 2.91 | 5.13 | 22.48 | 25.66 |
| | chlorophyll | 29.78 | 32.39 | 29.35 | 15.97 | 14.74 |
| | content after | | | | | |
| | deducting | | | | | |
| | water/% | | | | | |
| | average | 30.51 | | | / | / |
| | chlorophyll | | | | | |
| | content after | | | | | |
| | deducting | | | | | |
| | water/% | | | | | |
| | chlorophyll | 0.546 | 0.619 | 0.607 | 0.522 | 0.507 |
| | yield/% | | | | | |
| | average yield/% | 0.591 | | | / | / |
| | chlorophyll | 95.93 | 96.77 | 95.09 | 88.65 | 89.23 |
| | extraction rate/% | | | | | |
| | average | 95.93 | | | / | / |
| | chlorophyll | | | | | |
| | extraction rate/% | | | | | |
| 1- | product | 3.09 | 3.14 | 3.20 | / | / |
| deoxynojirimycin | content/% | | | | | |
| | product yield/% | 3.19 | 2.82 | 2.63 | / | / |
| | average product | 2.88 | | | / | / |
| | yield/% | | | | | |

Based on the results shown in Table 1, the following conclusions can be drawn:
(1) The chlorophyll paste in the examples had a moisture-adjusted content ranging from 29.35% to 32.39%, with an average content of 30.51%. This represents an improvement of 91.05% compared to Comparative Example 1 and 106.99% compared to Comparative Example 2;
(2) The chlorophyll extraction yield of the examples ranged from 95.09% to 96.77%, with an average yield of 95.93%. This represents an 8.21% improvement over Comparative Example 1 and a 7.51% improvement over Comparative Example 2;
(3) Example 3 and Comparative Examples 1 and 2 used the same batch of fresh silkworm excrement. A comparison shows that Example 3 achieved a chlorophyll paste yield of 0.607%, which is a 16.28% improvement over Comparative Example 1 and a 19.72% improvement over Comparative Example 2;
(4) Examples 1-3 employed mixed solvent extraction and subsequent processing steps to simultaneously obtain two products: chlorophyll paste and a 1-deoxynojirimycin product with a 3% content. The average yield of the 1-deoxynojirimycin product was 2.88%. By contrast, the comparative examples yielded only chlorophyll paste as a single product.

Based on the above analysis results, it can be concluded that: the process described in the present invention has clear advantages in terms of chlorophyll paste extraction yield, product yield, product content, and the comprehensive utilization of silkworm excrement. The chlorophyll paste produced by this invention can be applied in the preparation of microencapsulated chlorophyll, oil-soluble chlorophyll copper, and chlorophyll copper sodium salt products. The 3%-content 1-deoxynojirimycin product can be used in the functional food industry and other related fields.

The preceding description of specific exemplary embodiments of the present invention has been provided for illustrative and explanatory purposes. These descriptions are not intended to limit the invention to the precise forms disclosed, and it is apparent that many modifications and variations can be made in light of the above teachings. The selection and description of these exemplary embodiments are intended to explain the principles of the invention and its practical applications, enabling those skilled in the art to implement and utilize the invention's various exemplary embodiments as well as numerous options and modifications. The scope of the present invention is intended to be defined by the claims and their equivalents.

1. A method for simultaneously preparing a chlorophyll paste and 1-deoxynojirimycin, comprising the following steps:
(1) drying a silkworm excrement in a shaded area until the silkworm excrement reaches a specific moisture content;
(2) placing the silkworm excrement into an extraction tank, adding an extraction solvent to obtain a mixture, and stirring the mixture with a water bath, filtering the mixture, the extraction solvent comprising both a non-polar solvent and a polar solvent;
(3) subjecting the mixture to a coarse filtration to obtain a crude filtrate;
(4) subjecting the crude filtrate to centrifugation, obtaining a chlorophyll-rich light phase and a 1-deoxynojirimycin-rich heavy phase; and
(5) undergoing fine filtration and concentration with the chlorophyll-rich light phase to obtain a chlorophyll paste, and subjecting the 1-deoxynojirimycin-rich heavy phase with fine filtration, concentration, formulation, and spray drying to obtain 1-deoxynojirimycin.

2. The method as claimed in claim 1, wherein the specific moisture content of the silkworm excrement after the drying process in step (1) is between 40% and 60%.

3. The method as claimed in claim 1, wherein, in step (2), a mass ratio of the non-polar solvent to the polar solvent is (1-10):1.

4. The method as claimed in claim 3, wherein, in step (2), the non-polar solvent is an ether solvent or an alkane solvent, and the polar solvent is an alcohol.

5. The method as claimed in claim 4, wherein, in step (2), the non-polar solvent is selected from the group consisting of petroleum ether, diethyl ether, methyl tert-butyl ether, and an alkane with 4-7 carbon atoms; and the polar solvent is selected from the group consisting of methanol, ethanol, and isopropanol.

6. The method as claims in claim 1, wherein, in step (2), a solid-liquid ratio of the silkworm excrement to the extraction solvent is 1:(2-20); a temperature of the water bath is 30-60° C.; and an extraction time is 0.5-5 hours.

7. The method as in claim 1, wherein, in step (5), the chlorophyll-rich light phase is concentrated under a temperature of 40-70° C. and a pressure of 0.02 to 0.09 MPa.

8. The method as in claim 1, wherein, in step (5), the 1-deoxynojirimycin-rich heavy phase is concentrated under a temperature of 40-100° C. and a pressure of 0.02 to 0.09 MPa, and a concentration endpoint is at a Brix value of 10-40%.

9. The method as in claim 1, wherein, in step (5), the 1-deoxynojirimycin has a content of over 3% with a yield of 2.88%; the chlorophyll paste has a content of over 27%, an extraction yield exceeding 95%, and an average yield of 0.59%.
